(19) United States
(12) Patent Application Publication
(10) Pub. No.: US 2025/0107995 A1
(43) Pub. Date: Apr. 3, 2025

(54) NAIL POLISH TOPCOAT COMPOSITION

(71) Applicant: L'OREAL, PARIS (FR)

(72) Inventors: Maham AYAZ, East Brunswick, NJ (US); Ramakrishnan HARIHARAN, Springfield, NJ (US); I-fan HSIEH, Scotch Plains, NJ (US); Sam MORRISON, Piscataway, NJ (US)

(73) Assignee: L'OREAL, PARIS (FR)

(21) Appl. No.: 18/374,859

(22) Filed: Sep. 29, 2023

(51) Int. Cl. A61K 8/895; A61Q 3/02

(52) U.S. Cl. CPC A61K 8/895; A61Q 3/02

(57) ABSTRACT

A composition for use on the nails is provided, comprising a solvent system that includes at least one volatile, non-aqueous polar solvent; a water-insoluble nitrocellulose polymer; a silicone organic hybrid polymer and an alkyd resin. Methods including those for use as a topcoat are also provided.

### TECHNICAL FIELD

The present invention relates to nail polish topcoat compositions, and specifically to nail polish topcoat compositions comprising certain silicone organic hybrid polymer and an alkyd resin.

### BACKGROUND

Consumers use nail polish to cosmetically enhance the appearance of their nails or protect the nails from the abuses found in their everyday environment. This can be accomplished using various coatings that include base coat, color coat, and/or topcoat where basecoat can provide adhesion on nails, color coats can enhance the color and a layer of topcoat can provide shine (gloss).

Overtime, nail polish applied to nails loses its shine (gloss). The present inventors have now found a way to not only provide high initial shine but also to provide durable shine retention.

### BRIEF SUMMARY

The present invention is directed to a nail polish topcoat composition (also referred to herein as “nail topcoat composition,” or just “composition”). The nail polish topcoat composition includes a solvent system. The solvent system includes at least one volatile, non-aqueous polar solvent. The nail polish topcoat composition further includes a water-insoluble nitrocellulose polymer as well as a silicone organic hybrid polymer formed from at least one monomer comprising carboxylic acid and at least one vinyl monomer. The nail polish topcoat composition further includes an alkyd resin.

The silicone organic hybrid polymer may be a copolymer of a vinyl acetate, a vinylsilicone, a C3-7 carboxylic acid and a vinyl C7-45 alkyl ester. Advantageously, the silicone organic hybrid polymer may be a Crotonic Acid/Vinyl C8-12 Isoalkyl Esters/VA/Bis-Vinyldimethicone Crosspolymer and/or be present in a concentration by weight in the nail topcoat composition that is from about 0.05% to about 2.5% by weight.

It may also be advantageous for an alkyd resin to be or include a phthalic anhydride/glycerol/glycidyl decanoate copolymer and/or be present in a concentration by weight in the nail topcoat composition that is from about 8% to about 13%.

It may also be advantageous for the composition to be substantially free of colorant and free of UV curable material.

It may also be advantageous for the at least one volatile, non-aqueous polar solvent to include at least one volatile acetate, such as ethyl acetate and butyl acetate, such as where these compounds are present in a concentration by weight such that a total concentration by weight of ethyl acetate and butyl acetate in the nail topcoat composition is from about 50% to about 90%.

It may also be advantageous for the nail topcoat composition to further include sucrose acetate butyrate and/or acetyl tributyl citrate.

It may also be advantageous for the water-insoluble nitrocellulose polymer to be nitrocellulose such as to be present in a concentration by weight in the nail topcoat composition that is from about 5% to about 15%.

According to certain embodiments, the nail polish topcoat composition may meet a plurality or even all of these above listed requirements.

According to another aspect of the invention, a method of treating a nail is provided. The method includes applying to the nail the above-described nail polish topcoat composition.

### BRIEF DESCRIPTION OF THE DRAWINGS

Implementation of the present technology is described, by way of example only, with reference to the attached FIGURE, wherein:

FIG. 1. Shows a graph of shine (gloss) at 60 degrees over dry abrasion cycles for various compositions including different concentrations of alkyd resin and/or silicone organic hybrid polymer.

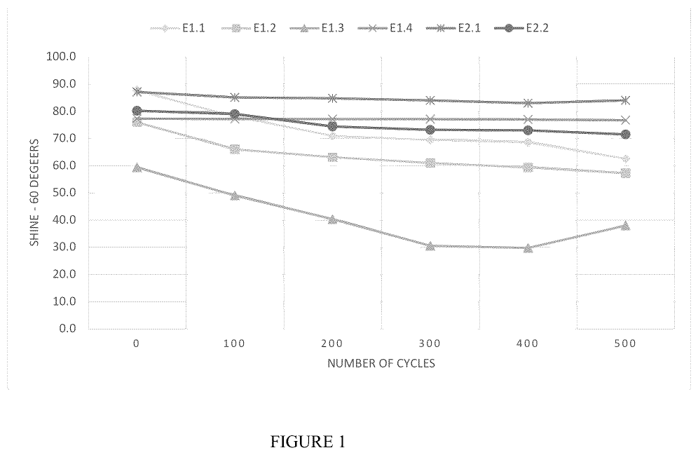

### DETAILED DESCRIPTION

As used herein, the expression “at least one” means one or more and thus includes individual components as well as mixtures/combinations.

As used herein, articles such as “a” and “an” when used in a claim, are understood to mean one or more of what is claimed or described.

As used herein, the terms “include”, “includes” and “including” are meant to be non-limiting.

Other than in the operating examples, or where otherwise indicated, all numbers expressing quantities (e.g., concentrations, ratios, and the like) of ingredients and/or reaction conditions are to be understood as being modified in certain embodiments by the term “about,” meaning within 10% to 15% of the indicated number (e.g. “about 10%” means 8.5% to 11.5% such as 9% to 11%, and “about 2%” means from 1.7% to 2.3 such as from 1.8% to 2.2%).

Similarly, for ratios, the modifier “about” means within 10% or 15% of the number. For example, about 4:1 means from 3.4:1 to 4.6:1, preferably 3.6:1 to 4.4:1. As readily understood by one skilled in the art, where the first ingredient in a ratio is less than the second, then a ratio may be expressed “inversely.” For example, if a second ingredient, B is present in an amount or concentration that is 2.5 times greater than that of ingredient A, this may be identified as an A:B ratio of 1:2.5. “About 1:10,” means from 1:8.5 to 1:11.5, preferably 1:9 to 1:11. Unless otherwise indicated, all concentrations shown as percentages are concentrations by weight and also, unless otherwise indicated, relate to the entire nail topcoat composition as a whole.

As used herein, the expression “active material” means with respect to the percent amount of an ingredient or raw material, refers to 100% activity of the ingredient or raw material.

“Film-former” or “film forming agent” or “film forming polymer” or “film forming resin” as used herein mean a polymer or resin that leaves a film on the substrate to which it is applied, for example, after a solvent accompanying the film-former has evaporated, absorbed into and/or dissipated on the substrate. A “silicone film-former” is a film-former that includes at least one silicone (a silicone atom bonded directly to an oxygen atom and also to a carbon atom such as in an organic moiety).

“Liquid” or “liquid cosmetic” or “liquid composition” means a composition having a fixed volume, flows to cover the bottom and assumes the shape of the portion of the container it fills and is slightly compressible.

“Substituted” as used herein, means comprising at least one substituent. Non-limiting examples of substituents include atoms, such as oxygen atoms and nitrogen atoms, as well as functional groups, such as amine groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Substantially free” as it is used herein means that while it is preferred that no amount of the specific component be present in the composition, it is possible to have small amounts of it in the compositions of the invention provided that these amounts do not materially affect at least one, preferably most, of the advantageous properties of the compositions of the invention. In certain embodiments, substantially free means less than about 2% of the identified ingredient, such as less than about 1%, such as less than about 0.5% based on the composition as a whole (or a particular component, if indicated). The term “anhydrous” means substantially free of water.

Numerical ranges are inclusive of endpoints and meant to include all combinations and sub-combinations. For example, from about 5%, 10% or 15% to about 20%, 50% or 60% may refer to about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%. As used herein a range of ratios is meant to include every specific ratio within, and combination of subranges between the given ranges.

“Shine” or “gloss” as used herein, refers to surface shininess. Gloss meters are commonly used in the nail polish art as well as in other areas of cosmetics and measure the amount of light reflected from the surface or film of interest. The gloss may be quantified, for example, as a percent reflectance at different angles specifically at 20°, 60°.

The compositions and methods of the present invention can comprise, consist of, or consist essentially of the essential elements and limitations of the invention described herein, as well as any additional or optional ingredients, components, or limitations described herein or otherwise useful.

Nail topcoat compositions of the present invention include a solvent system, a water-insoluble nitrocellulose polymer; a silicone organic hybrid polymer and an alkyd resin. Each of these will be discussed in turn.

### Solvent System

Nail topcoat compositions of the present invention include a solvent system. The function of the solvent system is to dissolve or disperse various components in the nail topcoat composition and to evaporate at an appropriate rate in order to allow for the relatively fast formation of film that has desirable shine and shine retention properties.

The solvent system includes at least one volatile, non-aqueous polar solvent. The volatile, non-aqueous polar solvent is (1) “volatile” in that it evaporates at a rate at least as fast as butyl acetate; (2) is polar in that it includes at least one polar functional group (e.g., alcohol, ether, ester, ketone, hydroxyl, amine, amino, carboxyl, carbonyl, and the like), and (3) “non-aqueous” in that it is not water, and, in certain embodiments is not completely miscible in water throughout all concentrations at standard temperature and pressure.

According to certain embodiments, the at least one volatile, non-aqueous polar solvent includes an ester, such as one or more acetate compounds. In particularly notable embodiments, the acetate compound is selected from ethyl acetate, propyl acetate, butyl acetate, and combinations thereof. In certain embodiments the one or more acetate compounds are used as a majority portion of the at least one volatile, non-aqueous polar solvent, meaning that the proportion by weight of the one or more acetate compounds in the total amount at least one volatile, non-aqueous polar solvent is more than about 50%, such as more than about 75%, such as more than about 90%.

According to certain embodiments, the at least one volatile, non-aqueous polar solvent is present in the nail topcoat composition in a concentration by weight that is from about 50% or 60% to about 75%, 80% or 90%. In notable embodiments, the at least one volatile, non-aqueous polar solvent comprises, has a majority fraction (and more notably at least about 90% of the total volatile, non-aqueous polar solvents) that is a mixture of volatile acetates such as ethyl acetate, and butyl acetate. In other notable embodiments, the nail topcoat composition has a total concentration by weight of ethyl acetate and butyl acetate in the nail topcoat composition is from about 50% to about 90%.

According to certain other embodiments the at least one volatile, non-aqueous polar solvent includes one or more monoalcohols, such as C2-C5 monoalcohol such as ethanol or isopropanol. In certain embodiments the one or more monoalcohols are used as a minority portion of the at least one volatile, non-aqueous polar solvent, meaning that the proportion by weight of the one or more monoalcohols in the total amount of the at least one volatile, non-aqueous polar solvent is less than 50%, such as less than about 25%, such as less than about 10%.

### Water-Insoluble Nitro Cellulose Polymer

Nail topcoat compositions of the present invention include a water-insoluble nitrocellulose polymer (e.g., cellulose nitrate). The nitrocellulose polymer may be formed by nitrating cellulose by exposure to acids such as nitric acid and/or sulfuric acid. Desirably the nitrocellulose is soluble in the solvent system and more particularly soluble in the one or more volatile, non-aqueous polar solvents.

The water-insoluble nitrocellulose polymer serves as a film-former in the nail topcoat composition. The nitrocellulose may be present in a concentration by weight in the nail topcoat composition that is from about 5%, 6% or 7% to about 10%, 12% or 15% by weight.

### Silicone Organic Hybrid Polymer

In accordance with the present invention, nail compositions comprising at least one silicone organic hybrid polymer are provided. A silicone organic hybrid polymer is formed from at least one monomer comprising carboxylic acid and at least one vinyl monomer. By “vinyl monomer,” as one skilled in the art will readily understand, it is meant a monomer of the formula H2C═CHR, wherein R represents a functional group, such as an H, an alkyl ester, or the like. Further, it is preferred that the at least one monomer comprising carboxylic acid and at least one vinyl monomer are two separate monomers. Preferably, the silicone organic hybrid polymer is selected from a silicone vinyl acetate compound and a crosslinked anionic copolymer comprised of organic polymer blocks and silicone blocks, resulting in a multiblock polymer structure, or mixtures thereof.

In particular, suitable examples of the silicone organic hybrid polymer of the present invention include, but are not limited to, copolymers of vinyl acetate and vinylsilicone. Such copolymers may optionally further include C3-7 carboxylic acid and/or vinyl C7-45 alkyl ester monomers.

In particular, suitable examples of the silicone organic hybrid polymer of the present invention include, but are not limited to, crosslinked anionic copolymers comprising at least one cross-linked polysiloxane structural unit. PCT patent application publication no. WO 2011/069786, the entire contents of which is hereby incorporated by reference, discloses examples of such compounds.

A preferred silicone organic hybrid polymer of the present disclosure is a compound having the INCI name of Crotonic Acid/Vinyl C8-12 Isoalkyl Esters/VA/Bis-Vinyldimethicone Crosspolymer which is a copolymer of Crotonic Acid, vinyl C8-12 isoalkyl esters and Vinyl Acetate crosslinked with bis-vinyldimethicone. This silicone organic hybrid polymer is commercially available from the company Wacker Chemie AG under the tradename Wacker Belsil® P1101 (may also be known under the tradename Wacker Belsil® P101) and may have an acid number from 28 to 33 mg KOH/g. Crotonic Acid/Vinyl C8-12 Isoalkyl Esters/VA/Bis-Vinyldimethicone Crosspolymer is also known by the technical name of Crotonic Acid/Vinyl C8-12 Isoalkyl Esters/VA/divinyldimethicone Crosspolymer.

Preferably, the at least one silicone organic hybrid polymer is present in the nail composition of the present invention in amounts of active material generally ranging from about 0.01% to about 5%, preferably from about 0.03% to about 3%, and more preferably from about 0.05% to about 2.5%, by weight, based on the total weight of the cosmetic composition, including all ranges and subranges in between.

### Alkyd Resin

In accordance with the present invention, nail compositions comprising at least one alkyd resin are provided.

Preferably, the at least one alkyd resin is a polyester comprising hydrocarbon chains of fatty acids. Such resins can be obtained by polymerization of polyols and polyacids or their corresponding anhydride in the presence of fatty acids, where the fatty acids can be employed “as is” or in the form of fatty acid triglycerides or in the form of oils during the synthesis of the alkyd resin. Due to the presence of hydrocarbon chains of fatty acids in the alkyd resin, alkyd resins are commonly defined by their oil length. Accordingly, “oil length of an alkyd resin” is understood to mean the percentage by weight of hydrocarbon chains of fatty acids present in the alkyd resin.

Examples of suitable polyols which can be employed in the synthesis of alkyd resin include, but are not limited to, at least one of pentaerythritol, trimethylolpropane, trimethylolethane, neopentyl glycol, propylene glycol, ethylene glycol, 1,6-hexanediol, 1,4-butanediol, diethylene glycol and, in particular, glycerol.

Examples of suitable polyacid or anhydride which can be employed in the synthesis of alkyd resins include, but are not limited to, at least one of, isophthalic acid, terephthalic acid, trimellitic anhydride, maleic anhydride, adipic acid, fumaric acid, azelaic acid, sebacic acid and, in particular, phthalic anhydride.

Examples of suitable fatty acids which can be employed in the synthesis of alkyd resins include, but are not limited to, at least one of fatty acids corresponding to the formula R—COOH, in which R denotes a saturated or unsaturated hydrocarbon radical preferably having from 7 to 45 carbon atoms, preferably from 9 to 35 carbon atoms, preferably from 15 to 35 carbon atoms and preferably from 15 to 21 carbon atoms. Mention may be made of, for example, palmitic acid, stearic acid, oleic acid, ricinoleic acid, linoleic acid, linolenic acid and, in particular, capric acid.

Fatty acids are present in the majority of oils of natural origin, in particular in the form of triglycerides. The triglycerides of fatty acids are esters resulting from the reaction of the three alcohol functional groups of glycerol with fatty acids, it being possible for these fatty acids to be identical or different. Oils of natural origin can thus be used during the polymerization. They can be chosen from, for example, linseed oil, China wood oil, oiticica oil, soybean oil, sunflower oil, safflower oil, castor oil, coconut oil, olive oil, palm oil, rapeseed oil, peanut oil and tall oil.

Specific examples of acceptable alkyd resins include, but are not limited to, at least one of those sold under the names “Alukidir ODE 230 70 E” or “Alukidir ODE 230 70 V” (phthalic anhydride/glycerol/glycidyl decanoate copolymer in ethyl acetate at 70%), “Necowel 581®” (50% in soybean oil), “Necowel 585®” (20% in sunflower oil), “Necowel 580®” (20% in sunflower oil), “Necowel 586 N®” (50% in soybean oil), “Necowel EP 1161®” (50% in soybean oil), “Necowel EP 1213®” (20% in oil), “Necowel EP 2009®” (32% in sunflower oil), “Necowel EP 2019®” (20% in oil), “Necowel EP 2275®” (35% in oil), “Necowel EP 2329®” (34% in oil), and “Necowel EP 3016®” (30% in oil) by Ashland or “Uradil XP 515 AZ®” (73% in tall oil) or “Uradil XP 516 AZ®” (63% in tall oil) by DSM Resins. “Alukidir ODE 230 70 E” or “Alukidir ODE 230 70 V” (phthalic anhydride/glycerol/glycidyl decanoate copolymer in ethyl acetate at 70%) is particularly preferred.

Preferably, the at least one alkyd resin is present in the nail composition of the present invention in amounts of active material generally ranging from about 0.5%, 0.7%, 0.8%, 0.9%, 1.0%, 1.5%, 2.0%, 2.5%. 3.0%, 3.5%. 4.0%, 4.5%. 5.0%, 5.5%, 6.0%, 6.5%, 7.0%, 7.5% or 8.0% to about 8.0%, 9.0% 10.0%, 11.0%, 12.0%, 13.0%. 14.0% 15.0%, 16.0%, 17.0%, 18.0%. 19% or 20% and more preferably from about 8% to about 13%, by weight, based on the total weight of the cosmetic composition, including all ranges and subranges in between. Further the at least one alkyd resin and the silicone organic hybrid polymer may be present in the nail composition in a ratio by weight from about 5:1, 6:1 or 7:1 to about 7:1, 10:1, 15:1, 20:1, 30:1, 40:1, 50:1, 60:1, 70:1, 80:1, 90:1, or 100:1.

### Other Ingredients

Nail topcoat compositions of the present invention may include various other ingredients without significantly compromising the beneficial properties.

For example, according to certain embodiments, plasticizers other than the alkyd resin may be included in the nail topcoat composition. Examples of such other plasticizers include polar molecules having multiple ester groups such as those having a molecular weight in a range from about 250 or 400 to about 1000 or 1500. Examples include sucrose acetate butyrate and acetyl tributyl citrate. Additional example of other plasticizer includes a cellulose ester of at least one C2-C4 organic acid. Examples include cellulose acetate butyrate. In certain embodiments the nail topcoat composition includes an ingredient selected from a group consisting of cellulose acetate butyrate, sucrose acetate butyrate and acetyl tributyl citrate. Cellulose acetate butyrate commercially available from Eastman Chemical such as CAB-381-0.5. Sucrose acetate butyrate is sold under the name SAIB 90 by Eastman Chemical of Kingsport, TN. Acetyl tributyl citrate is sold under Citroflex brand by Vertellus, or the Uniplex brand by Unitex Chemical of Greensboro, North Carolina. These other plasticizers may be present in concentrations ranging from about 1%, 2% or 3% to about 10%, 15% or 25% by weight.

Other additional exemplary plasticizers include dipropylene glycol dibenzoate, and 1,2-propylene glycol dibenzoate, trioctyl citrate, triethyl citrate, acetyl tributyl citrate, or tributyl citrate. These other additional plasticizers may be present in concentrations ranging from about 1%, 2% or 3% to about 10%, 15% or 25% by weight.

As another example, according to certain embodiments, additional polymers or resins may be included in the nail topcoat composition. One example of a suitable additional polymer or resin are acrylate copolymers. An example of a suitable acrylate copolymer is “acrylate copolymer”, having an acid number that ranges from about 145 and about 170 mg KOH per gram such as EPOMATT G-152, EPOMATT G-154 or ISOCRYL G-265 available from Estron Chemical, Inc of Calvert City, KY. Additional potential acrylates copolymers include to those sold under the PECOREZ® name such as, for example, PECOREZ AC 50 available from Phoenix Chemical.

Another example of a suitable polymer or resin is polyester resins formed by reaction of a polyhydric alcohol with a basic polyacid, for example phthalic acid, such as the commercial product sold by Unitex Chemical Corporation under the name UNIPLEX 670-P, which is a polyester resin obtained by reaction of trimellitic acid, neopentyl glycol and adipic acid. (Meth)acrylic resins, according to the disclosure, can include copolymers of methyl methacrylate with butyl acrylate, butyl methacrylate, isobutyl methacrylate or isobornyl methacrylate, for example the commercial products PARALOID DM-55, PARALOID B48N, PARALOID B66 and ELVACITE 2550; copolymers of isobutyl methacrylate and butyl methacrylate, for example, the commercial product ELVACITE 2046; and isobutyl methacrylate polymers, for example, PARALOID B67.

These additional polymers or resins may be present in concentrations ranging from about 1%, 2% or 3% to about 10%, 15% or 25% by weight.

As another example, according to certain embodiments, mattifying agents may also be included in the nail topcoat composition. Examples include silica, aluminosilicates, borosilicates, and the like. These mattifying agents may be present in concentrations ranging from about 0.25%, 0.5% or 1% to about 1%, 2% or 5% by weight.

In certain notable embodiments, the nail polish topcoat composition may include certain components such as water and/or colorants (coloring agent). However, in other embodiments, it is generally preferred that the nail topcoat composition be substantially free of water and/or colorants.

The nail topcoat composition may be substantially free of dyes, pigments, pearls, effect pigments and the like that are commonly used in color cosmetics or in color coat nail polishes.

Dyes include, for example, fat-soluble dyes such as Sudan red, DC Red 17, DC Green 6, β-carotene, Sudan brown, DC Yellow 11, DC Violet 2, DC Orange 5 and quinoline yellow.

Pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2.

Pearlescent pigments include, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and pearlescent pigments based on bismuth oxychloride.

Nail topcoat compositions of the present invention may be made by methods known in the art, such as by optionally premixing certain ingredients into the one more solvent and blending various ingredients together in careful manner to provide a homogeneous mixture.

### Methods of Use

Nail topcoat compositions may be applied onto a nail such as human fingernail or toenail. According to certain embodiments the nail has a previously applied color coat formed thereon and the nail topcoat composition is applied on top of the previously applied color coat formed on the nail. The nail topcoat composition may be applied onto the nail/previously applied color coat formed thereon by commonly used methods such as brushing or spraying.

The following examples are intended to illustrate the invention without limiting the scope as a result. The percentages are given on an active weight basis.

### Examples: Composition and Testing

### Example 1: Comparative Examples

The compositions below in Table 1 (Comparative Examples) were prepared to evaluate the shine retention of topcoat compositions after exposure to various abrasion test conditions specifically dry abrasion, wet abrasion, and soap abrasion. These comparative compositions contained varying amounts of alkyd resin. Specifically, three compositions were prepared having different concentrations of alkyd resin, in addition the fourth composition was prepared having a silicone organic hybrid polymer without alkyd resin.

### Example 2: Inventive Examples

The compositions below in Table 2 (Inventive Examples) were prepared to evaluate the shine retention of topcoat compositions after exposure to various abrasion test conditions specifically dry abrasion, wet abrasion, and soap abrasion. These compositions contained varying amounts of alkyd resin with silicone organic hybrid polymer. Specifically, two compositions were prepared having the different concentrations of alkyd resin with silicone organic hybrid polymer.

### Sample Preparation

Samples were prepared by combining solvents, nitrocellulose, silicone organic hybrid polymer, alkyd resin, other plasticizers, other film-forming polymers, and other ingredients in a jar. The blend was allowed to let sit 12 to 24 hours. It was then mixed for three minutes with overhead mixer until uniform. The samples were then allowed to sit for 24 hours before testing so any air bubbles would dissipate. The test nail topcoat compositions were evaluated to measure shine retention by measuring shine (gloss) at 20° and 60° after exposure to various abrasion test conditions specifically dry abrasion, wet abrasion, and soap abrasion.

### Test Description

To measure shine retention, a film is drawn using a 6-mil drawdown bar onto the BYKO-chart Black Scrub Panel P121-10N and allowed to dry for 24 hours under ambient conditions. The Black Scrub Panel was placed in BYK Gardner-scrub machine, base model. An abrasive BYK Gardner 8812 brush and BYK 500 g weight was used to perform abrasion test under various conditions specifically dry abrasion, wet abrasion, and soap abrasion. After 100 cycles, wipe the surface of Scrub Panel with dry wipe and measure the shine (gloss) using BYK-Gardener Micro-Tri-Glossmeter. Repeat the same procedure until 500 cycles.

TABLE

| | Comparative Examples - Table 1 | | | | Inventive Examples - Table 2 | |
|---|---|---|---|---|---|---|
| Components | E1.1 | E1.2 | E1.3 | E1.4 | E2.1 | E2.2 |
| Solvents, including acetates | 72.3 | 78.5 | 77.1 | 77.1 | 72.3 | 78.5 |
| Nitrocellulose | 9.5 | 9.6 | 11.2 | 11.2 | 9.5 | 9.6 |
| Crotonic acid/vinyl C8-12 | ** | ** | ** | 0.1 | 0.1 | 0.1 |
| isoalkyl esters/VA/bis- | | | | | | |
| vinyldimethicone | | | | | | |
| crosspolymer | | | | | | |
| Phthalic | 9.9 | 0.7 | ** | ** | 9.9 | 0.7 |
| anhydride/glycerol/glycidyl | | | | | | |
| decanoate copolymer. | | | | | | |
| Other film forming | 2.0 | 6.5 | 5.7 | 5.7 | 2.0 | 6.5 |
| polymers | | | | | | |
| Other plasticizers | 6.2 | 4.8 | 6.0 | 6.0 | 6.2 | 4.8 |
| *Apparent errors due to | | | | | | |
| rounding | | | | | | |
| Total | 100 | 100 | 100 | 100 | 100 | 100 |
| 60-degree shine | | | | | | |
| 0 Cycles | 87.6 | 75.9 | 59.3 | 77.2 | 87 | 80.1 |
| 100 Cycles | 78.1 | 66 | 49 | 77.1 | 85 | 78.9 |
| 500 Cycles | 62.6 | 57.2 | 37.9 | 76.6 | 83.9 | 71.4 |
| Δ Shine (0 Cycle-500 | 25 | 18.7 | 21.4 | 0.6 | 3.1 | 8.7 |
| Cycle) | | | | | | |
| 20-degree shine | | | | | | |
| 0 Cycles | 58.8 | 31.8 | 18.3 | 34 | 57.9 | 38.1 |
| 100 Cycles | 39.4 | 23 | 11.1 | 33.5 | 52.3 | 37.3 |
| 500 Cycles | 21.6 | 14.4 | 3.5 | 32.7 | 48.7 | 29 |
| Δ Shine (0 Cycle-500 | 37.2 | 17.4 | 14.8 | 1.3 | 9.2 | 9.1 |
| Cycle) | | | | | | |

### Result

The nail topcoat compositions and shine (gloss) results are set forth in Table 1 & Table 2 above.

According to the results in Table 1 & Table 2, the composition with a silicone organic hybrid polymer improved the shine retention and the compositions with both a silicone organic hybrid polymer and an alkyd resin clearly improves the initial shine & shine retention under dry abrasion conditions. The effect is particularly pronounced at alkyd concentrations greater than 0.7% by weight, such as shown in Example 2.1.

What is claimed is:

1. A composition for use on nails, comprising:
a solvent system comprising at least one volatile, non-aqueous polar solvent;
a water-insoluble nitrocellulose polymer;
a silicone organic hybrid polymer formed from at least one monomer comprising carboxylic acid and at least one vinyl monomer; and
an alkyd resin.

2. The composition of claim 1, wherein the silicone organic hybrid polymer is a copolymer of a vinyl acetate, a vinylsilicone, a C3-7 carboxylic acid and a vinyl C7-45 alkyl ester.

3. The composition of claim 1, wherein the silicone organic hybrid polymer is a Crotonic Acid/Vinyl C8-12 Isoalkyl Esters/VA/Bis-Vinyldimethicone Crosspolymer.

4. The composition of claim 1, wherein the silicone organic hybrid polymer is free of phenyl moieties.

5. The composition of claim 1, wherein the alkyd resin is a phthalic anhydride/glycerol/glycidyl decanoate copolymer.

6. The composition of claim 1, wherein the silicone organic hybrid polymer is present in a concentration by weight in the composition that is from about 0.05% to about 2.5% by weight.

7. The composition of claim 1, wherein the alkyd resin is present in a concentration by weight in the composition that is from about 8% to about 13%.

8. The composition of claim 1, wherein the composition is substantially free of colorant.

9. The composition of claim 1, wherein the composition is substantially free of UV curable material.

10. The composition of claim 1, wherein the at least one volatile, non-aqueous polar solvent comprises at least one volatile acetate.

11. The composition of claim 1, wherein the solvent system comprising at least one volatile, non-aqueous polar solvent comprises ethyl acetate and butyl acetate each in a respective concentration by weight such that a total concentration by weight of ethyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein silicone organic hybrid polymer is Crotonic Acid/Vinyl C8-12 Isoalkyl Esters/VA/Bis-Vinyldimethicone Crosspolymer and wherein Crotonic Acid/Vinyl C8-12 Isoalkyl Esters/VA/Bis-Vinyldimethicone Crosspolymer is present in a concentration by weight in the composition that is from about 0.05% to about 2.5% by weight, and wherein the alkyd resin is phthalic anhydride/glycerol/glycidyl decanoate copolymer and wherein phthalic anhydride/glycerol/glycidyl decanoate copolymer is present in a concentration by weight in the composition that is from about 8% to about 13%.

12. A method of treating a nail, comprising;
applying to the nail a composition comprising:
a solvent system comprising at least one volatile, non-aqueous polar solvent;
a water-insoluble nitrocellulose polymer;
a silicone organic hybrid polymer formed from at least one monomer comprising carboxylic acid and at least one vinyl monomer; and
an alkyd resin.

13. The method of claim 12, wherein the nail has a previously applied color coat formed thereon and the composition is applied on top of the previously applied color coat formed on the nail.

14. The method of claim 12, wherein the silicone organic hybrid polymer is a copolymer of a vinyl acetate, a vinylsilicone, a C3-7 carboxylic acid and a vinyl C7-45 alkyl ester.

15. The method of claim 12, wherein the silicone organic hybrid polymer is a Crotonic Acid/Vinyl C8-12 Isoalkyl Esters/VA/Bis-Vinyldimethicone Crosspolymer.

16. The method of claim 12, wherein the alkyd resin is a phthalic anhydride/glycerol/glycidyl decanoate copolymer.

17. The method of claim 12, wherein the silicone organic hybrid polymer is present in a concentration by weight in the composition that is from about 0.05% to about 2.5% by weight.

18. The method of claim 12, wherein the alkyd resin is present in a concentration by weight in the composition that is from about 8% to about 13%.

19. The method of claim 12, wherein the composition is substantially free of UV curable material.

20. The method of claim 12, wherein the solvent system comprising at least one volatile, non-aqueous polar solvent comprises ethyl acetate and butyl acetate each in a respective concentration by weight such that a total concentration by weight of ethyl acetate and butyl acetate in the composition is from about 50% to about 90%, wherein the water-insoluble nitrocellulose polymer is nitrocellulose and wherein the nitrocellulose is present in a concentration by weight in the composition that is from about 5% to about 15%, wherein silicone organic hybrid polymer is Crotonic Acid/Vinyl C8-12 Isoalkyl Esters/VA/Bis-Vinyldimethicone Crosspolymer and wherein Crotonic Acid/Vinyl C8-12 Isoalkyl Esters/VA/Bis-Vinyldimethicone Crosspolymer is present in a concentration by weight in the composition that is from about 0.05% to about 2.5% by weight, and wherein the alkyd resin is phthalic anhydride/glycerol/glycidyl decanoate copolymer and wherein phthalic anhydride/glycerol/glycidyl decanoate copolymer is present in a concentration by weight in the composition that is from about 8% to about 13%.
